(19) United States
(12) Patent Application Publication
(10) Pub. No.: US 2004/0166550 A1
(43) Pub. Date: Aug. 26, 2004

(54) Bio-gateway

(76) Inventors: Brian M. Sullivan, Manhattan Beach, CA (US); Denes L. Zsolnay, Rolling Hills Estates, CA (US)

Correspondence Address: RONALD M. GOLDMAN, ROTH & GOLDMAN, SUITE 500, 21535 HAWTHORNE BLVD., TORRANCE, CA, 90503, US

(21) Appl. No.: 10/373,937

(22) Filed: Feb. 25, 2003

(51) Int. Cl. G01N 33/53

(52) U.S. Cl. 435/7.94

(57) ABSTRACT

Bioagent surveillance of airline passengers is accomplished by having the passenger walk through a gateway (1) to be exposed to a stream of air (3) that sweeps away any bioagents exhaled by the passenger and/or struck from the passenger's clothing. The swept air, including any bioagents, is collected (5, 7) and tested (9) for the presence of any of a predefined list of bioagents; and the presence of a bioagent displayed (10).

### REFERENCE TO PRIOR APPLICATIONS

Reference is made to U.S. application Ser. No. 09/837,946, filed Apr. 19, 2001, entitled “Automated Computer Controlled Reporter Device for Conducting Immunoassay and Molecular Biology Procedures,” to TRW Docket No. 15-0236, entitled “Charged Bio-Molecule Binding Agent Conjugate for Biological Capture” and to application Ser. No. 10/055,318, filed Oct. 23, 2001, entitled Combinational Strategy for Identification of Biological Agents, all of which are assigned to the assignee of the present application.

### FIELD OF THE INVENTION

This invention relates to bioagent surveillance apparatus, and, more particularly, to method and apparatus for screening airline passengers and/or other individuals for pathogens and other bioagents.

### BACKGROUND

One often hears that airline passengers readily contract the common cold on board commercial aircraft. That anecdotal evidence points to the aircraft cabin as a fertile place for transmission of infectious airborne disease. One reason for that popular belief is the industry's practice of recirculating cabin air. To reduce consumption of fuel and cost of operating the aircraft, the policy of the airlines is to recirculate a large percentage of the air in the aircraft interior, drawing in fresh air for the remainder. In that process, the recirculated air is filtered to remove all impurities and pathogens from the cabin air. Based on experience, airline passengers are not confident of the effectiveness of that filtering.

Many frequent fliers experience discomfort on debarking long flights. Because passengers are usually closely packed together, there is little doubt that transmissible pathogens expressed by an adjacent passenger are likely to find their way into the air that one breathes in. An ill passenger moving up and down the narrow isles to the lavatory, similarly, will likely leave a trail of atomized pathogens that may reach other passengers. The wide dispersal is particularly likely if the isles are crowded, with other passengers walking to and from the lavatories.

For most, the common cold is annoying, but not a major health threat. The slight and short-term debilitation and ennui caused by the common cold may be considered an acceptable cost of airline travel. However, other infectious diseases, such as the flu or tuberculosis, pose major health threats and cannot be so easily dismissed. Although the spread of airborne pathogens may be reduced by drawing a greater percentage of outside air into the aircraft cabin, expelling a greater percentage of used air from the aircraft cabin, no credible proof exists that doing so would significantly decrease the spread of pathogens aboard the aircraft, although the comfort of the passengers would certainly be enhanced. Arguably, the increased cost attendant to supplying more fresh air inside the cabin could price airline travel beyond the reach of many. Hence, the air recirculation practice persists of apparent necessity.

To protect the health of passengers, the airlines have the right to refuse passage to a passenger that is visibly sick with a serious infectious disease. In making that refusal airline personnel must be willing to incur some risk to their personal career, if their non-medical diagnosis latter proves incorrect. That potential liability provides a powerful disincentive for action. Moreover, even if visibly sick persons are consistently excluded, the problem persists because an infected passenger may not exhibit visible symptoms of disease at the time of boarding. In sum, apart from the anecdotal evidence, credible information concerning the health risks posed by sick air passengers is lacking. Further, there has been no way to determine which passengers are or may be causing a health problem for other passengers (or the pilots of the aircraft). To serve the health of the flying public, there is a need to obtain data on infectious diseases that potentially could be carried onboard commercial aircraft as well as to identify and/or quarantine those infected disease-carrying passengers.

One theoretical solution is to require each passenger to undergo a Doctor's examination just prior to boarding the aircraft. Such a solution is obviously impractical, if not absurd, being intrusive, unduly time consuming and expensive. Further, the effectiveness of such a doctor's examination is questionable and is dependent upon the experience and/or expertise of the individual doctor. While most will have much experience with the major diseases, very few are likely to be versed in diagnosing all infectious diseases of concern to the airlines. As an advantage, the initial embodiment of the present invention provides a practical solution to identifying diseasey-carrying passengers.

Airport security was recently enhanced as a result of the quadruple aircraft hijackings on Sept. 11, 2001. To prevent weapons or dangerous materials on board an aircraft, all passengers and their carry-on baggage are searched at airport security using one form of search or other. Some of those searches are by hand and others are by electronic equipment. Further, the baggage loaded on the aircraft for a flight must be matched to a boarding passenger on that flight. The effect of the efforts to bar a few evil-doers from boarding an aircraft is visible: long lines of passengers enduring the long delays in passing through airport security. When those bedraggled passengers arrive at the gate, they take comfort in the belief they are safe. However, the security check does not uncover or offer any security from bioagents, even though some bioagents may be more lethal as a weapon than an explosive. As a further advantage, the present invention extends airport security to a check for disease or other bioagents as would give the passengers more reason to feel safe.

Beyond basic surveillance, one should also recognize and take into account practical necessities, such as passenger inconvenience. Passengers are already faced with long lines and delays due to the enhanced airport security. Ideally, any bioagent surveillance system should not impede the progress of the passenger to the gate or otherwise add to the frustration and delay of air travel. Unfortunately, testing of bioagents as presently accomplished requires some time to complete. Hence, the initial embodiment of the invention requires the passenger to wait at security until that testing is completed. Should the passenger be permitted to proceed to the gate before testing for bioagents is completed some way is needed to identify that passenger from amongst the many passengers usually found at the airport gate.

As a further advantage, an advanced embodiment of the present invention extends airport security to a check for disease or other bioagents and can do so without increasing passenger delays beyond that now produced by existing security checks. As a further advantage, the advanced embodiment takes advantage of existing baggage security technology to identify the passenger at the gate.

Airports come in many sizes and shapes. Despite that variety, all airports contain at least one security checkpoint and at least one gate. Others, such as Los Angeles International airport, contain many security check points, each of which leads to a large number of gates. In a small airport with only one gate, should security later find need to question a passenger after the passenger has been passed through security, the passenger may easily be located. Because of the multiplicity of gates in the large airport, locating that passenger is difficult, or near impossible. From the security standpoint alone, locating a passenger who earlier passed through security is beneficial.

In order to cross check baggage against a boarding passenger, it is necessary for the passenger to be given and retain some form of identification, such as a baggage number applied to his ticket. That information is inputted in the management information computer system for the airport. The airport representative at the gate is then able to check the list of checked baggage numbers on his data display against the baggage number on the passenger ticket. If the passenger has not checked baggage, then the passenger would be assigned a no-checked baggage number by airport security. If any checked baggage cannot be matched against a boarding passenger, then the baggage will be destroyed.

Because of the security measures, all passengers are made to carry some form of identification issued at the airport. Should a question arise concerning any piece of checked baggage, the passenger may be located by checking those identification numbers. One may recall the one aircraft bombing attempt some years ago by an anti-western Arab militant who planted a bomb in the luggage of his English girlfriend, bidding his lady a fond farewell on her flight to the U.S. The bomb was detected in the checked luggage; the passenger was quickly located, and led to the apprehension of the criminal. As a still further advantage advanced embodiments of the present invention are able to take advantage of the security baggage to passenger identification system.

Accordingly, an object of the present invention is to protect the health of airline passengers.

Another object of the invention is to prevent infectious diseases or bioagents from being carried on board commercial aircraft by the passengers.

A further object of the invention is to identify airline passengers who are infected with or carry undesired bioagents before the passenger is permitted to board the aircraft.

A further object of the invention is to provide the means to inspect airline passengers for disease or other bioagents with little or no intrusion of the person and without the need to require the presence of a physician.

A still further object of the invention is to enable data to be gathered inconspicuously and unobtrusively on infectious diseases borne by airline passengers.

And an additional object of the invention is to provide bioagent surveillance of airline passengers without unduly interfering with the passenger's progress to the passenger's aircraft gate.

### SUMMARY OF THE INVENTION

In accordance with the invention, a surveillance region through which airline passengers must pass comprises a gateway. That gateway includes a blower to direct a stream of air onto a passenger passing through the gateway, which, deflected from the passenger, carries away any bioagents exhaled in the passenger's breath and/or that is struck from the passenger's clothing; means to collect the air deflected from the passenger, including any bioagents carried by that deflected air; and means to receive and test that collected air for the presence of any of a predefined list of bioagents. In accordance with a specific aspect of the invention, the means to test comprises a computer controlled automated testing apparatus that performs an enzyme linked immunoassay (“ELISA”) process. Further, in accordance with another specific aspect of the invention, the floor of the gateway comprises a grate, and the stream of air is provided from a location vertically above the passenger and passes through the gate to the automated testing apparatus.

In a more technologically advanced embodiment of the invention means are included to employ the baggage security number or other number assigned by security as the passengers identification, and to associate that passenger identification with a bioagent test, whereby the passenger is permitted to proceed to the gate before the testing is completed, yet can be later identified at the gate as needed. In accordance with a specific aspect a bank of individual bioagent testing devices are employed to run the tests. As each passenger moves through the gateway, one of those bioagent testing devices is assigned to run the test for bioagents; and that testing device continues through to completion of the test, even though that passenger earlier exited the gateway. The next passenger through the gate is assigned to a different testing device, and that testing device may overlap in operation with the testing device assigned to the preceding passenger. With the advanced embodiment, passengers are not delayed, and, although the passenger proceeds to the gate, bioagent security is unimpaired.

The foregoing and additional objects and advantages of the invention, together with the structure characteristic thereof, which were only briefly summarized in the foregoing passages, will become more apparent to those skilled in the art upon reading the detailed description of a preferred embodiment of the invention, which follows in this specification, taken together with the illustrations thereof presented in the accompanying drawings.

### BRIEF DESCRIPTION OF THE DRAWINGS

In the drawings:

FIG. 1 illustrates an embodiment of the invention;

FIG. 2 is a block diagram of another embodiment of the invention; and

FIG. 3 illustrates a valving system used with the embodiment of FIG. 2.

### DETAILED DESCRIPTION OF THE PREFERRED EMBODIMENTS

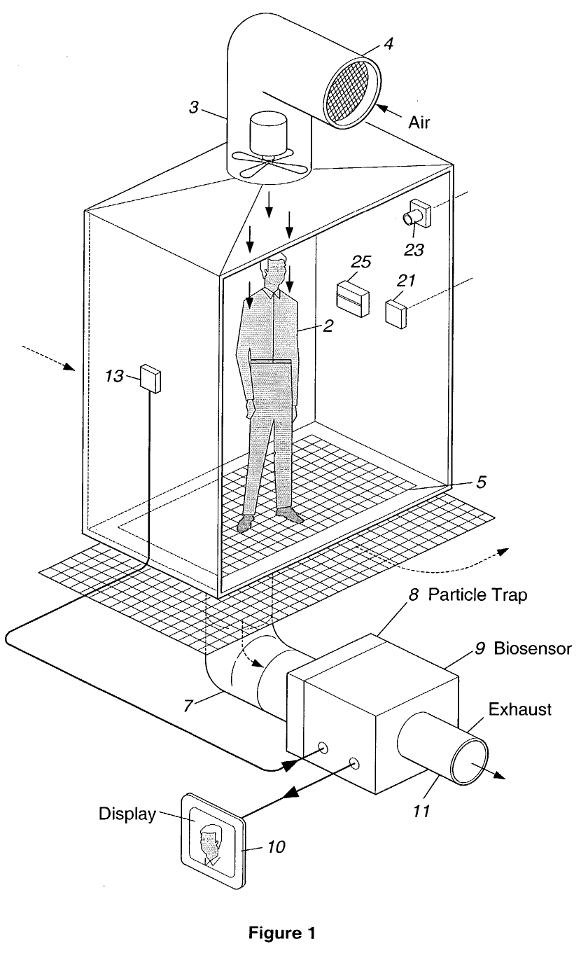

Reference is made to FIG. 1, which pictorially illustrates the bioagent surveillance system of the invention. The surveillance system includes a defined region for temporarily receiving passengers, such as a room, chamber or gateway 1; a blower unit or fan 3, located on the top side or ceiling to the gateway; a grate 5 on the bottom that serves as the floor or walkway inside the gateway; an air duct 7; a particle trap 8 and a biosensor unit 9 that is automated in operation. Ducting 7 may be built into the floor of the airport or within the basement region of the airport that underlies the passenger walkway, so that portion of the apparatus is not visible to the passenger. The biosensor unit includes a display, such as LCD display 10 on which to display test results. A power switch 3 is included to enable the system operator to energize biosensor unit 9. The air output of biosensor 9 is ducted by a duct 11 to the exhaust system in the airport, not illustrated. Additional elements illustrated, but not presently described, are used in connection with an additional embodiment of the invention and are described later in this specification.

In this embodiment, gateway 1 contains an open entrance and exit. The gateway is positioned in the walkway of the airport, preferably at a position immediately following the normal electronic security checkpoint so that the passenger 2 must immediately pass through gateway 1. This forces the passenger to pass through both security and bioagent gateways, effectively in one “swoop,” minimizing irritation of the passenger. Overhead, fan 3 is ducted 4 to an external air supply, such as the airports air distribution system, from which to draw air.

When the system is operating, fan 3 may be continuously powered from an external source, not illustrated. Passenger 2 is required to enter gateway 1 from security and is sufficient in size to receive the passenger and the passenger's carry-on baggage. The fan draws air through the duct and blows that air down over the passenger 2 and into the openings in grate 5. The openings in grate 5 lead to duct 7, which underlies the grate. The air and any particulate matter entering duct 7 passes into the particle trap 8 and, from there, passes into biosensor unit 9.

Particle trap 8 is an electrically operated device of known structure that traps and removes large particulate material from the air, cleaning the air of dust, rubber particles, insect debris and the like before the air enters biosensor 9. That particulate matter is not detected by the biosensor and any accumulation of that particulate in the biosensor would only serve to mechanically stress or clog the biosensor. As a good housekeeping measure that debris is best removed. Although some such particulate matter may remain in the air following the particle trap, as becomes apparent, the operation of the biosensor apparatus includes some washing steps, which should remove any such residue.

Particle traps of the foregoing type are marketed by the Midwest Research Institute as the SPINCON cyclone particle generator. Briefly the SPINCON generator comprises an open glass cylinder with a vertical slit cut at an angle through the cylindrical wall. Suction is applied to the exit end and the inlet air to the other. Water is introduced from a reservoir to the exterior of the cylinder while air enters the cylinder through the slit, producing a stable swirling motion of water about the inner cylindrical wall. Small particles impact the wall due to the inertia of the particles and become suspended in the fluid. The fluid is removed by discontinuing the vacuum and allowing gravity to pull the fluid to the bottom of the cylinder, where the fluid is sucked out and introduced to the biosensor.

Biosensor 9, later herein discussed in greater detail, is a computer controlled automated unit that is capable of inspecting the incoming air for the presence of predetermined bioagents. That is, the biosensor may be programmed to be on the look-out for, that is, sense, one particular bioagent or a group of selected bioagents in the entering air. A biosensor of this type is described in our copending U.S. application Ser. No. 09/837,946, filed Apr. 19, 2001, entitled “Automated Computer Controlled Reporter Device for Conducting Immunoassay and Molecular Biology Procedures” (the “946 application”), assigned to the assignee of the present application, the content of which is incorporated herewithin by reference.

As explicitly noted in the cited 946 application, the automated tester is capable of detecting the presence within a suspect solution of a bioagent from within a predetermined group of bioagents. Although the description in that application seeks to identify a single specific bioagent by introducing into the suspect solution in the initial step of the testing process an antigen for the specific bioagent, it is apparent that several different antigens could be introduced into the suspect solution simultaneously, each of which is specific to a different bioagent (thereby defining a group consisting of three bioagents, for example). Then the ELISA test is able to identify the presence of any of the bioagents of that group in the suspect solution. That approach is the one used in the present invention. The group of antibodies is selected to include all the bioagents of concern to the airline passenger. The automated test determines whether any of those diseases is present. That identification is essentially a “go” or “no-go” procedure. At this stage of the health security check, there is no need to identify the particular bioagent that was detected and caused the alarm, only that a bioagent is present.

Bioagents may originate on the clothing or baggage and may be exhaled by the passenger in the passenger's breath. For example, the clothing of the passenger could possibly contain anthrax spores. The passenger may be sick with any of flu, tuberculosis, smallpox, botulism, tularensis, ebola, lassa fever or encephalitis and exhale the bacteria or virus of the respective disease in the passenger's breath.

In operation of the foregoing embodiment, passenger 2 is directed to enter the gateway 2. In compliance, the passenger walks along the walkway and enters the defined region of that gateway. The system administrator or automated passenger detector, not illustrated, operates switch 13 to energize biosensor 9 and particle trap 8. On entering, the passenger is brushed by the downward flow of air directed from fan 3. That air is incident on the passenger, hitting the passenger on the head, on the passenger's clothing and any hand carried baggage, and then passes downward through the openings in grate 5 and into duct 7. Some of the air blown by the fan flows around the passenger and also into the openings in grate 5. Effectively, the airstream produced by fan 3 sweeps any bioagent in its path through the openings in the floor grate.

The air and the bioagent carried by the air, along with any debris, streams through the grate, travels along duct 7 and into particle trap 8, where the airstream is cleaned of the debris. In addition to any relevant bioagent as may be drawn into the automated system inlet 4, the air swept into the inlet also contains the particulate matter normally found in the air around the airport, particularly a major urban airport like Los Angeles. That particulate includes diesel soot, dirt, tire rubber, acid and the like, collectively referred to herein as debris. The recognition molecules of the ELISA process performed by biosensor 9, as later herein described, do not “link” to that debris. Particle trap 8 removes the majority of that debris, and any remainder is removed during one or more of the washing steps as is typically included in the automated system of biosensor 9. From the trap 8 the resulting fluid and any bioagents carried therein is directed into biosensor 9.

While the passenger exits the gateway and waits, the automated biosensor 9 runs the test looking for the presence of a predetermined group of bioagents in the suspect solution. That predetermined group of bioagents may include, for example, anthrax, flu, tuberculosis, smallpox, botulism, tularensis, Ebola, lassa fever and encephalitis. On completion of the test, biosensor 9 produces a “pass” or “fail” designation for the passenger that appears on display 10. The passenger is appropriately treated by the operator and associated security personnel in accordance with the particular indication displayed. The system may then be reset to accommodate the next passenger. Biosensor 9 is an automated test for the presence of bioagents. One such apparatus that tests for biogens automatically uses the enzyme linked immunoassay (“ELISA”) procedure is described, for one, in the 946 application.

The ELISA process uses molecular interactions to uniquely identify target substances. A basic definition of ELISA is a quantitative in vitro test for an antibody or antigen (e.g., a bioagent) in which the test material is adsorbed on a surface and exposed to a complex of an enzyme linked to an antibody specific for the substance being tested for with a positive result indicated by a treatment yielding a color in proportion to the amount of antigen or antibody in the test material, or, as recently expanded in definition, by an electrical current in proportion to the amount of antigen or antibody in the test material. The basic ELISA procedure is described more specifically, for one, in a book entitled, Methods in Molecular Biology, Vol. 42, John R. Crowther, Humana Press, 1995.

In the ELISA test for a suspect bioagent carried out by the automated apparatus of the '946 application, the suspect bioagent is initially placed in a water-based buffer, such as a phosphate buffered saline solution, to form a sample solution. That sample solution is mixed with a quantity of magnetic beads, the surface of which are coated with an antibody to the suspect bioagent, sometimes generically referred to as a recognition molecule or receptor molecule. The foregoing antibodies, called a primary antibody or “1° Ab,” are known to bind to the bioagent of interest or of concern, exhibiting a chemical “stickiness” that is selective to specific bioagents.

If the suspect bioagent is present in the sample solution, the bioagent forms a non-covalent bond to a respective antibody and, hence, is attached to a respective one of the magnetic beads. If the sample solution does not contain a bioagent or if the bioagent that is present in the solution is not one that binds to the selected antibody, then no binding occurs and further processing of the ELISA process will show nothing. With the suspect bioagent present, the solution then contains a quantity of bioagent molecules bound respectively to a like quantity of coated magnetic beads. The mixture is optionally washed, for example, in a phosphate-buffered saline, and a second antibody, more specifically, an antibody and enzyme-linked combination, is then added to the mixture.

The second antibody is also one that is known to bind to the suspect bioagent, another recognition molecule. The second antibody is either one that is monoclonal, e.g., one that binds to only one specific molecule, or polyclonal, e.g., a mixture of different antibodies each of which shares the characteristic of bonding to the target bioagent. The enzyme is covalently bound to the second antibody and forms a complex that is referred to as a secondary antibody-enzyme conjugate or “2° Ab-enz.” As is known, an enzyme is a “molecular scissors,” a protein that catalyzes a biological reaction, a reaction that does not occur appreciably in the absence of the enzyme. The enzyme is selected to allow the subsequent production of an electrochemically active reporter.

The 2° Ab-enz binds to the exposed surface of the bioagent to form an “antibody sandwich” with the bioagent forming the middle layer of that sandwich. The antibody sandwich coated beads are washed again to wash away any excess 2° Ab-enz in the solution that remains unbound. The magnetic beads and the attached antibody sandwich, the 1° Ab/bioagent/2° Ab-enz complex, in the solution are positioned in the solution over the exposed surface of a sensor, such as a redox recycling sensor. The substrate of the foregoing enzyme is then added to the solution and the substrate is cleaved by the enzyme to produce an electrochemically active reporter. The substrate of the enzyme, referred to as PAP-GP, is any substance that reacts with an enzyme to modify the substrate. The effect of the enzyme is to separate, cut, the PAP, a para-amino phenol, the electrochemically active reporter, from the GP, an electrochemically inactive substance. That chemical cleaving reaction is concentrated at the surface of the sensor.

The rate of production of the foregoing reporter (PAP) is proportional to the initial concentration of bioagent. The reporter reacts at the surface of the sensor, producing an electrical current through the sensor that varies with time and is proportional to the concentration of the bioagent, referred to as redox recycling. The occurrence of the electric current constitutes a positive indication of the presence of the suspect bioagent in the sample. Analysis of the electric currents produced over an interval of time and comparison of the values of that electric current with existing laboratory standards of known bioagents allows quantification of the concentration of bioagent present in the initial sample.

The foregoing automated testing apparatus employs magnetic fields to manipulate the position of the magnetic beads and is but one example of an automated tester. Another employs electrically charged recognition molecules and an electric field to manipulate the position of those charged molecules, such as described in our copending application, TRW Docket No. 15-0236, entitled “Charged Bio-Molecule Binding Agent Conjugate for Biological Capture.” For additional details of the construction of the automated testing apparatus, the reader should refer to the cited applications, which are incorporated herein by reference.

Although the foregoing example given of testing was for a single suspect bioagent, the apparatus may also be used to search for a group of different suspect bioagents. That is accomplished by including several different recognition molecules in the test, for example, by coating the magnetic beads used in the automated tester of the '946 application with multiple recognition molecules or by coating groups of magnetic beads with recognition molecules for different bioagents and employing a mixture of those groups in the test. The foregoing procedure is described and employed in another of our copending U.S. patent application Ser. No. 10/055,318, filed Oct. 23, 2001, entitled Combinational Strategy for Identification of Biological Agents. Thus if automated testing is to test for any of eight different bioagents, then specific antibodies for each of those bioagents is included.

In the foregoing embodiment of FIG. 1, gateway 1 contained an open entrance and exit, enabling the passenger to walk in and then walk right out without the bother of opening and closing doors. In other embodiments, the entrance and exits of gateway 1 may contain closures, such as automatic doors, to form a closed room while the passenger is proceeding from entrance to exit. Although that is more complicated in structure, a closed room minimizes the introduction of extraneous airborne particles while the passenger is being inspected by the apparatus.

An additional and still more complicated variation of the gateway is to employ a revolving door, and to limit the air current produced by the overhead fan to one sector of that door. When the particular sector of the door that contains the passenger is rotated by the passenger to a predetermined angular position, the air current flows from the top to the bottom of that sector to sweep the passenger. Like the conventional closure, with the revolving door, a closed region is defined once that door rotates to the predetermined angle, and thereby limits the introduction of extraneous air and particulate matter during the test. All of the foregoing alternative structures provide an inspection region that may be partially or fully confined when the inspection of the passenger is made. It should be appreciated from an understanding of the invention that all of the alternatives are regarded as equivalent.

Existing technology for the biosensor 9 does not allow a test for the selected bioagents to be completed instantaneously or within the short interval in which the passenger is situated inside gateway 1. The duration of the test (e.g., the test interval) is on the order of minutes. Hence, unless each passenger is to be detained at the biogateway until the test results arrive as in the preceding embodiment, by the time the test result is delivered by the apparatus, the passenger should be on his or her way to the gate at which his or her flight is scheduled to depart. Although the foregoing embodiment accomplishes the desired surveillance, the passenger is required to stand by until the automated testing is completed and the result displayed.

Detention of passengers at the biogateway would create another long line, equal or greater in length to that occurring at the entrance to the customary security station, and additional inconvenience for the passenger to a degree that might render such surveillance politically unacceptable. Such a situation is best avoided, and, accordingly, additional and more complex embodiments of the invention, next considered, include features to avoid that difficulty. The embodiment permits the passenger to exit and progress to the aircraft gate before the test result is delivered by the biosensor. As becomes apparent from the following description, the passenger is not required to remain at gate 1 and a passenger can be located at the aircraft gate, and, if necessary, be prevented from boarding the aircraft.

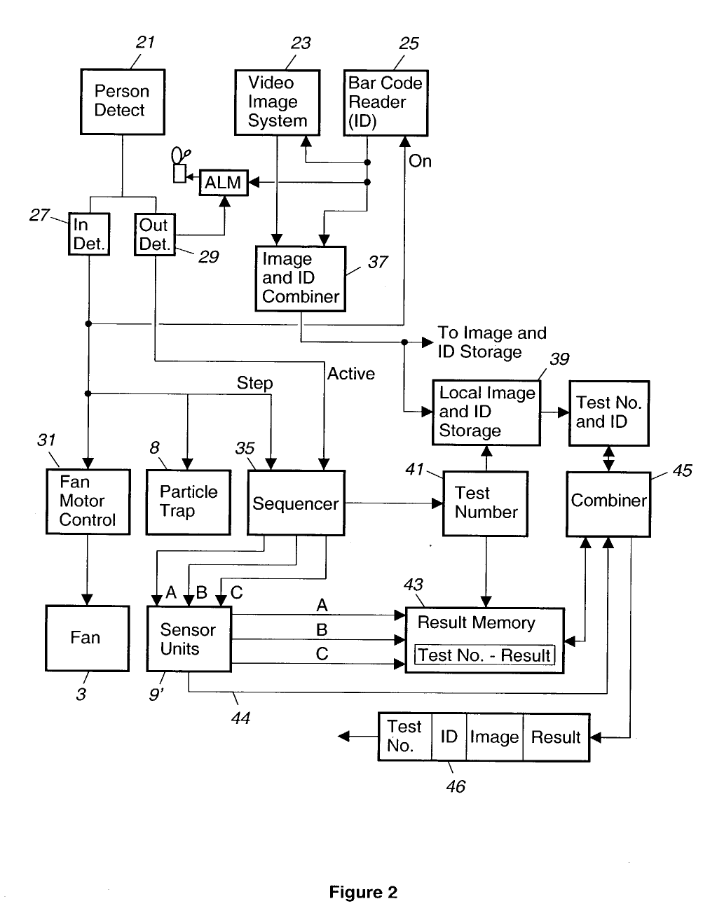

Reference is made to FIG. 2, which shows a control and information system in block diagram form. That control and information system is combined with the gateway embodiment of FIG. 1 to form a system that doesn't require the passenger to linger at the gateway. Instead of a single biosensor 9, this embodiment contains three separate biosensors, not separately illustrated in FIG. 2, collectively designated 9′ and individually identified by letters A, B and C. Those biosensors operate independently of one another, and, generally will overlap in operation, as hereafter described. The system includes a personnel detector 21 to detect the presence of a passenger inside gateway 1, a video camera for recording the image of the passenger 23 in a computer readible data file, such as the JPEG file type, and a bar code reader 25 to read the identification data, later described, given to the passenger. The foregoing devices are mounted in a side wall of the gateway and are visible to the passenger as illustrated in FIG. 1 to which the reader may briefly refer. Since most of the structures of FIG. 1 are employed in this embodiment, for convenience, that illustration will also be referred to in this second embodiment and any modifications in the structure necessitated will be described.

Continuing with FIG. 2, detector 21 functions in conjunction with two electronic circuits, entry detector 27 and exit detector 29 to signal respectively the entry or exit of the passenger from the gateway. For example, the personnel detector may be of the infra-red type that detects reflection of infra-red energy from the person and changes a voltage output from a low to a high. Entry detector 27 detects the rise of the voltage, interpreting that change as an entry, and produces an output signal, while exit detector detects the drop of output from the high to a low, interpreting that change as an exit, and produces an output signal.

In operation the entry of the passenger into the gateway is thereby detected and entry detector 27 produces an entry detection signal. That signal is coupled to a control input of the fan motor control 31, which, in turn, energizes electrically powered fan 3; is also coupled to the control input of particle trap 8, also electrically operated as earlier discussed, which operates; and is coupled to bar code reader 25, which prepares to receive a card swipe of the passenger's identity card. The entry detection signal is also applied as an input to a sequencing device, sequencer 35. The sequencer activates the various biosensor units in a serial order and then repeats that loop with additional activation.

Fan 3 operates and blows air onto the passenger, sweeping any bioagent expelled from or knocked off the passenger through grate 5, where the bioagents are ducted 7 into particle trap 8. Assuming sequencer 35 is in idle condition pointing to biosensor C, when next activated by entry detector 27, the sequencer steps to or signals the next biosensor A in the sequence, and triggers operation of the respective biosensor. The automated biosensor includes a latch-up circuit. Once activated, the respective biosensor latches in the active state and commences operation, remaining in operation until the ELISA test process has been completed, and thereupon resets to an idle condition awaiting another trigger signal from sequencer 35.

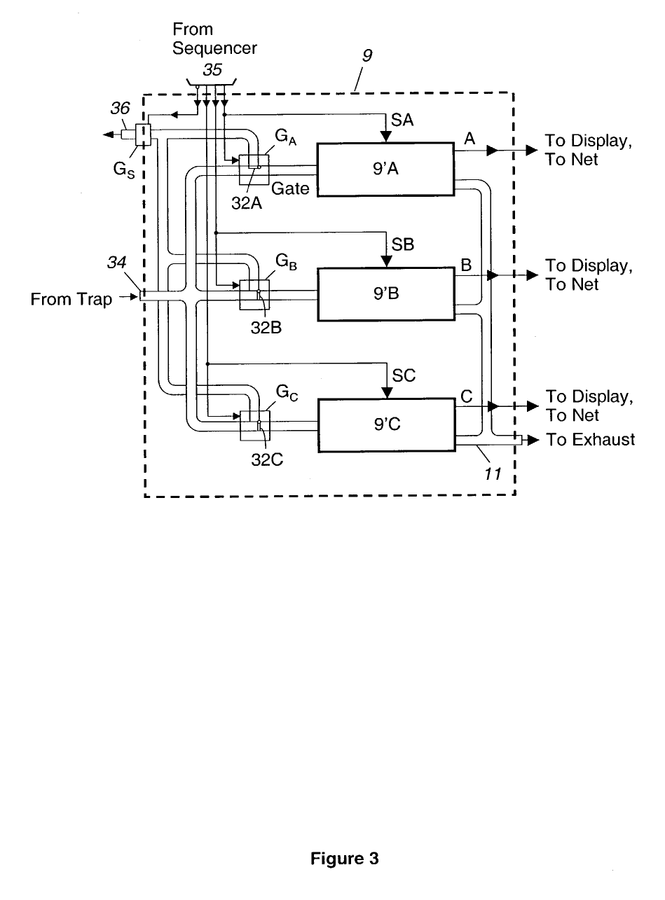

Sequencer 35 includes a transport mechanism which, on the lapse of a predetermined interval, sufficient to allow time for the sweeping action of the fan 3 to blow any bioagent from the passenger through the grate 5 and into the particle trap, and for the particle trap to output the bioagent in a solution through a fluid gating circuit to the selected one of the biosensors 9′ A, B, or C. A timing circuit, not illustrated, included in the biosensor 9′, delays operation of the initial step of the automated test of the biosensor by a slightly greater interval than that provided for the transport mechanism to ensure that the transfer of the suspect solution has been completed. A suitable transport mechanism is illustrated in FIG. 3, later herein described.

In addition to triggering the start of a biosensor 9′, sequencer 35 also signals a test number device 45, which in response produces a unique test number for the biosensor test. That device may be a simple electronic counter whose output count changes with each input from the sequencer providing a binary count that serves as the test number. The test number generated is supplied to memory 39, where the test number is associated with the image and ID data of the particular passenger in the gateway obtained from the video and card swipe. That test number is also supplied to the RESULT memory 43.

When the passenger looks at card reader 25 and swipes the numbered security ID card through the card reader, the card reader in response initiates or triggers the imaging system 23 and sends the security data read from the card into the image and ID combiner 37. The imaging system snaps a digital picture of the passenger combined with the ID number that was inputted through the card reader and provides that digital picture (as a JPEG data file, for example) to image and ID combiner 37, which associates the two pieces of data. That image file is identified or addressed in the combiner 37, for example, by the security ID code. When the test number is applied to and read into memory 39, the image and ID data of the passenger from combiner 37 is then copied to a local memory 39, where that data is indexed against the test number. The copied image and ID data may also be backed up at a remote storage, such as hard disk storage.

The test number is also applied to the result device 43 and is associated with an output of the respective biosensor, 9′ A, B or C, that is running the test just initiated by sequencer 35. That is, the test number is placed in a memory location that is associated with biosensor A and contains space in which to include the positive or negative result information from the respective biosensor when the biosensor has concluded its test of the bioagents. When that test is concluded the memory data contains the test number and the test result. As earlier described, the automated test for bioagents takes some time to complete, much greater than the time required to snap the picture of the passenger. By the time the test is completed, the passenger has already exited gateway 1 and has likely reached the gate. Result memory 43 acts as a buffer between the rapidly obtained imaging information and the more slowly obtained test information for a particular passenger that is supplied some minutes later.

The records from storage 39 are indexed by test number and contain the ID and Image data associated with the passenger. The result memory 43 is also indexed by test number. Each time a biosensor completes its test, the test completion is signaled, not illustrated, via 44 to a combiner 45. Prompted by that signal, the combiner then searches for the three most recent test numbers in the index of the result memory 43. When a match is found, the data for that entry in the result memory, the test result, is transferred or copied into the combiner. Combiner 45 combines that received data for records previously received from storage 39 having the same index number (e.g., test number) and provides a complete record containing the test number, the ID, the passenger image and the ELISA test result, such as illustrated at 46. Accordingly, the later supplied information now catches up with the other information earlier obtained. As those skilled in the art recognize combiner 45 is preferably formed of a programmed microprocessor. That output data may be sent to external networks that are used by the gate agents of the airlines and/or may be network accessible from the display terminals used by those gate agents.

The data 46 may be resorted or indexed by identification number. At the gate, the operator has available a list of baggage identification numbers (or psuedo-baggage numbers) and checks for passenger information for that baggage ID number. Alternatively, the gate agent may check through the list of passengers and look at the test results. Should one of the passengers be found to have a negative result (e.g., the displayed attribute blinks), the agent will request the passenger with a specified ID number to proceed to the gate and ask for the agent. When the passenger shows the ID ticket, and the number is confirmed, the agent may advise the passenger of the testing that transpired.

For example, “Mr. Jones, during your trip through security you were tested for the presence of any of the diseases or other bioagents on this list (handing the list to Mr. Jones). You tested positive. Unfortunately our testing does not identify which of the listed diseases produced the positive indication, and it's not likely to be one of the more serious ones. However, Federal law does not permit a passenger who tests positive to board the aircraft. Because automated testing is not always correct, to be certain we'd like to run a more complete test on you. We invite you to proceed to room 5079, where an individual test can be performed. So you should hurry if you want to see if you are able to board this flight. If the test is negative, then you'll be allowed to board the airplane. Otherwise we cannot let you board. Will you agree to take the additional test?”

Should Mr. Smith decline or fail the test, his baggage can be identified and removed from the aircraft. With a positive result, the ticket agent should always advise the passenger to immediately see a physician experienced in infectious diseases, before having the passenger escorted from the airport or turn the passenger over to the local health authorities for possible quarantine.

The list of bioagents range in seriousness from those that are not immediately life threatening, such as the flu, to others such as Ebola, which could strike terror in the heart of the passenger (and, perhaps, the gate agent as well). On viewing the disease list, a passenger might collapse, suffer shortness of breath or chest pains in reaction as the passenger contemplates the threat to his or her mortality. The airport counseling service should be contacted and requested to stand-by.

While one test is proceeding, a succeeding test of another passenger may commence. Once sequencer 35 is stepped to the next available biosensor, the sequencer is inhibited from subsequent activation, until an input is received at the ACTIVE input. When a passenger exits gateway 1 (FIG. 1), the departure is recognized by the departure detector 29, which signals the ACTIVE input of sequencer 35 of the departure. With that input to sequencer 35, the inhibit is removed from the sequencer, and the sequencer is then prepared to receive and respond to a signal from entry detector 27.

Assume another passenger enters gateway 1. The circuits operate as described previously to enable the fan 3, the particle trap 8 and so on, which operation need not be repeated in its entirety. The entry detection signal from detector 27 is input to the STEP input sequencer 35, and, in response, the sequencer switches or steps to the next available biosensor in the bank of biosensors 9′. Since the first biosensor employed in this description was 9′A, the sequencer steps to biosensor 9′ B. Biosensor 9′B operates in exactly the same way earlier described, which need not be repeated. It is noted that biosensor 9′ A may be finishing up one test, while biosensor 9′ B begins running a test.

Assume the foregoing action is occurring. When the second passenger departs the gateway, that passengers test number from 41 is associated in the result memory 43 with the output of biosensor 9′B. Assuming next that biosensor 9′ A completes the test, the biosensor posts the result, positive or negative, in result unit 43 at the address for the test number earlier assigned for the test. The result for the test number is then combined in combiner 45 at the address for the test number with the identification number. What results for each is a line of data that contains a test number, an identification ID number, an image address for the image of that customer stored in memory and the test result.

FIG. 3 to which reference is made, illustrates a bank of three biosensors, 9′A, 9′B and 9′C, that comprise biosensor unit 9′ in the embodiment of FIG. 2, and the valve system for delivering the solution containing the bioagent from the output of trap 8 to the automated biosensor selected by sequencer 35.

Each biosensor contains a power input SA, SB, and SC through which a start signal or power is applied, and, in response to that signal or power, the respective biosensor commences performance of the test (e.g., performing an ELISA process) on the gas or liquid solution that is supplied to the biosensor inlet. The output data or signal from the biosensors, indicated as A, B and C, respectively, is connected to a display, not illustrated in the figure, such as described in connection with the embodiment of FIG. 1. And the exhaust of each biosensor is connected in parallel to provide the exhaust 11 of the biosensor unit as in FIG. 1.

The biosensor unit contains and three electric gate valves Ga, Gb and Gc, one for each biosensor unit, and a stop valve GS. Each gate valve includes a gate 32a, 32b and 32c, respectively, that contains an inlet and two outlets. A duct 34, which couples to the particle trap earlier described, is connected to the inlet end of the gate valves, illustrated at the left, in parallel. The first outlet of each gate valve connects to the inlet of a respective one of the biosensors, 9′A, 9′B and 9′C, and the second outlet of those valves are connected in parallel to an exhaust conduit 36.

The gate of the respective gate valve controls whether the valve inlet is connected through to the first outlet or to the second outlet. Normally the gate of the gate valve blocks the first outlet and opens the second outlet when the gate valve is not energized, such as illustrated for gates 32B and 32C. When the gate valve is energized, such as by an electrical input from sequencer 35, the valve moves the gate to open the first outlet and block the second outlet, such as illustrated for gate 32A. Each gate contains an input through which electrical power to energize the valve may be applied. Those inputs are coupled to respective outputs of the sequencer 35, earlier described, that supply the electrical power in sequence to those gates and to the associated biosensor unit. The sequencer concurrently supplies power to gate valve Gs to close the normally open valve to prevent the solution from flowing out the exhaust duct 36 to a sump, not illustrated, until sufficient solution has been pumped through the open gate valve and into the input to the respective automated biosensor.

If the sequencer 35 doesn't supply power to a gate and associated biosensor, any solution introduced is bypassed through the gate valves and through stop valve G2 into the exhaust duct 36. Assuming sequencer 35 supplies power to stop valve Gs, gate valve GA and the SA input of the associated biosensor 9′ A, stop valve Gs closes, blocking the exhaust duct, gate GA energizes and opens the passage to the first outlet, and the biosensor commences operation. From the first outlet of gate valve GA, the test solution is introduced to the input of the biosensor 9′A. The biosensor acts on that solution and after the lapse of an interval, produces an output at A to the display.

Each automated biosensor contains appropriate power “holding” circuits, not illustrated, which continue to supply operating power to the biosensor, once the biosensor is started, and resets, when the biosensor completes operation. Thus, once the biosensor commences operation, the biosensor continues to completion, although the start signal at SA is removed by the sequencer 35.

When the testing interval is quite long, the sequencer 35 will advance to the next position, removing the power from gate GA and biosensor 9′A, and, for example, energizes gate GB and biosensor 9′B, even while biosensor 9′A continues the testing procedure. Thus, biosensor 9′B will commence operation concurrently.

It is believed that the foregoing description of the preferred embodiments of the invention is sufficient in detail to enable one skilled in the art to make and use the invention without undue experimentation. However, it is expressly understood that the detail of the elements comprising the embodiment presented for the foregoing purpose is not intended to limit the scope of the invention in any way, in as much as equivalents to those elements and other modifications thereof, all of which come within the scope of the invention, will become apparent to those skilled in the art upon reading this specification. Thus, the invention is to be broadly construed within the full scope of the appended claims.

What is claimed is:

1. A method of non-intrusively detecting the presence of any of a predetermined group of bioagents on or in a person, comprising the steps of:
applying an airstream to the person for sweeping bioagents exhaled by the person or which are present on the clothing of the person downstream;
collecting at least a portion of said airstream from downstream; and
testing said portion for the presence of any of said predetermined group of bioagents.

2. The method of non-intrusively detecting the presence of any of a predetermined group of bioagents on or in a person as defined in claim 1, wherein said step of applying an airstream to the person for sweeping bioagents exhaled by the person or which are present on the clothing of the person downstream, further comprises the steps of:
directing said person to enter and walk through a housing, said housing having an upper side, a walkway, said walkway including passages therethrough, and a blower for blowing an airstream from said upper side of said housing vertically downward toward said walkway, whereby said airstream strikes the head and clothing of said person when said person is present in said housing and exits through said walkway; and
wherein said step of collecting at least a portion of said airstream from downstream, further comprises the step of:
collecting at least a portion of said airstream from downstream under said walkway.

3. The method of non-intrusively detecting the presence of any of a predetermined group of bioagents on or in a person as defined in claim 1, wherein said step of testing said portion for the presence of any of said predetermined group of bioagents, further comprises the step of:
depositing said portion in an automated ELISA apparatus, wherein said automated ELISA apparatus automatically tests said portion for the presence of said predetermined group of bioagents.

4. The method of non-intrusively detecting the presence of any of a predetermined group of bioagents on or in a person as defined in claim 1, further comprising the steps of:
assigning a digital identification number to the person; and
producing a digital photograph of the person.

5. The method of non-intrusively detecting the presence of any of a predetermined group of bioagents on or in a person as defined in claim 4, further comprising the steps of:
assigning a test number to said testing; and
associating said test number with said digital identification and said digital photograph and saving said test number, digital photograph and said digital identification number in a memory.

6. The method of non-intrusively detecting the presence of any of a predetermined group of bioagents on or in a person as defined in claim 1, further comprising the step of:
displaying the result of said step of testing said portion for the presence of any of said predetermined group of bioagents.
